(19) United States
(12) Patent Application Publication
(10) Pub. No.: US 2016/0367384 A1
(43) Pub. Date: Dec. 22, 2016

(54) USER ADJUSTABLE PROSTHETIC ANKLE THAT COMPENSATES FOR DIFFERENT HEEL HEIGHTS

(71) Applicants: Carter H. Sigmon, San Diego, CA (US); Brian A. Zalewski, San Clemente, CA (US); Darwin Clark, Coronado, CA (US); The United States of America as Represented by the Secretary of the Navy, Silver Spring, MD (US)

(72) Inventors: Carter H. Sigmon, San Diego, CA (US); Brian A. Zalewski, San Clemente, CA (US); Darwin Clark, Coronado, CA (US)

(73) Assignee: The United States of America as represented by the secretary of the Navy, Silver Spring, MD (US)

(21) Appl. No.: 15/090,047

(22) Filed: Apr. 4, 2016

Related U.S. Application Data
(60) Provisional application No. 62/143,078, filed on Apr. 4, 2015.

(51) Int. Cl. A61F 2/66

(52) U.S. Cl. CPC A61F 2/6607; A61F 2002/5039

(57) ABSTRACT

A prosthetic ankle adapted to be attached to the leg and foot components of a prosthetic leg that allows user to self-adjustment of the pitch angle of the prosthetic leg via a turnbuckle-like mechanism to accommodate the heel heights of different shoes.

### CROSS-REFERENCE TO RELATED APPLICATION

This application claims priority to U.S. Provisional Application No. 62/143,078, filed on Apr. 4, 2015.

### BACKGROUND OF THE INVENTION

The present invention is related to ankle prosthesis for amputees, and particularly to an ankle designed to compensate for the pitch angle induced by different heel heights of the shoes worn in combination with prosthetic leg.

The lower extremity of prosthetic devices for both below-knee and above-knee amputees is conventionally a rigid heel to allow the amputee a relatively firm control over the foot while walking. Because tremendous dynamic forces applied to the prosthetic components during the simple act of walking, the sculptured foot component is often securely fixed to the leg component to form a rigid integral structure. Even minor misalignment may lead to obvious problems of balance and safety, an adjustment mechanism must be strong and avoid excessive wear or loosening that would affect stability. Customarily, the alignment and adjustment of the foot component is performed with the participation of the amputee prior to final shaping and bonding to the leg or ankle block. The resulting prosthetic leg component and foot component are permanently fixed to prevent subsequent misalignment. The initial alignment and adjustment is done based on the heel height of a particular shoe, and the amputee must continue to wear footwear that has identical heel heights. However, heel height can vary dramatically depending on the footwear. For example, the angle between the heel and the toe is negative for ski boots and is positive for high heeled shoes. The angle between the leg prosthesis's longitudinal axis and the horizontal axis is typically referred to as a pitch angle, which is also called inclination angle. When a different shoe is worn, the pitch angle changes and the stability of the prosthesis is affected, which can result in unnatural gait, or an unsafe limb. While the leg component can be subsequently dismantled, realigned and adjusted for a different heel height in a clinic, the user is limited to proscribed prosthetic legs with fixed angles.

The advantage of prosthesis with user adjustable heel height is readily appreciated. Many attempts has been made to develop a leg prosthesis that allows for adjustable heal height. Some designs address the issue with adjustable mechanisms located below the leg component, within the prosthetic foot component. For example, Patent U.S. Pat. No. 4,306,320 to Delp describes an adjustment mechanism works by sliding an upper assembly along the top of a wedge beneath the foot to compensate for the height of a shoe heel. The metal parts of this mechanism are located at or just above the shoe tops. U.S. Pat. No. 4,413,360 to Lamb, discloses an integrated foot-and-ankle design, with the adjustment mechanism placed within the prosthetic foot. The mechanism employs a pivot, which is associated with an adjustment screw that can alter the fore and aft tilt of the foot block with respect to the ankle block. Patent U.S. Pat. No. 5,913,901 to LaCroix employs a complex metal ankle joint prosthesis that comprises a hollow shaft concentric with an ankle axis which is pivotally mounted in a shaft bore for rotating said upper and lower ankle parts relative to each other about the ankle axis, which also include a semi-circular splined locking mechanism. The unit has over a dozen metal parts that extend to the heel of the prosthetic foot. These designs add a significant weight to the prosthetic foot component, which is located at the farthest end of the prosthetic leg. This mass and distance combination creates a strong momentum against the swing motion of walking, which the user must compensate. Acting as a pendulum, the user must exert additional efforts to swing the prosthetic leg forward, and then halt it to stop to start and complete each stride. A lighter design located further up the prosthetic leg is clearly more desirable.

A smaller number of products seek to provide adjustable ankle functionality with a device integral to the prosthetic leg component. Most of them employ motors, servos and batteries to automatically correct the pitch angle. However these designs tend to be heavy and expensive and often require special configuration, which is not compatible with other prosthetic components. U.S. Pat. No. 5,800,564 to Gelineau, describes a mechanical design, which uses a pair of matching swivel disks located at the end of the prosthetic leg component held together by a clamping bolt allow adjustment in 5 degree increments. However, this design not only limits the angles available to the user, and can be at times hazardous. If the clamping bolt came loose, the ankle would fall into two parts and render the user unable to stand or walk. Patent U.S. Pat. No. 8,641,780 to Abimosleh uses two hydraulic cylinders, connected by a valve, to change and hold positions of the ankle unit. Failures in the valves and seals are going to render the ankle unit inoperable and require special maintenance beyond the capability of most EMCs. Furthermore, both designs significantly increase the prosthetic leg's mass, which contributes to a swinging gait and compensation by the user.

A few products aim at a modular ankle unit that can interface with different prosthetic leg designs. Patent U.S. Pat. No. 8,628,585 to Harris discloses mechanism which uses a cylinder to dampen movement and provide movement to the shin area above the ankle unit. Although may be compatible with, the Harris design still add significant weight to the prosthetic leg and requires a unique design for the acritical foot.

### SUMMARY OF THE INVENTION

It is an object of the present invention to provide a prosthetic ankle that readily allows the user to adjust the pitch angle of a prosthetic leg to accommodate the heel height of different shoes.

Another object is to provide a prosthetic ankle that can be adjusted to accommodate a wide range of pitch angles.

Another object is to provide such a prosthetic ankle that is relatively light in weight, sturdy, and economical, and achieves the above and other objects and advantages without using heavy cylinders, hydraulic fluids, or mechanical valves.

Another object is to provide a prosthetic ankle that possesses the foregoing advantages and is adaptable for use with other prior art prosthetic leg designs.

Yet another object is to provide a prosthetic leg that comprises of a shin/leg component, foot component and prosthetic ankle of the present design.

According to the first aspect of this invention, the prosthetic ankle achieves pitch angle adjustment through a mechanical turnbuckle design, without the need of heavy cylinders, hydraulic fluids, or mechanical valves, which reduces the overall weight of the prosthetic leg and patient efforts required for each stride. Because the present prosthetic ankle is connected between the leg component and the foot component at a higher location on the prosthetic leg, the swinging momentum caused by the prosthetic ankle assembly is greatly decreased, and need for a compensating gait also reduced. Unlike some other designs, the present invention can support a wide range of pitch angles, which is the angle between the longitudinal axis of the prosthetic leg (axis of the shin component) and the horizontal axis of the prosthetic leg (axis of the foot component), to compensate for different heel heights. Once fixed, the design does not provide a flexing motion in the shin or ankle. The invented adjustable prosthetic ankle assembly thus provides a reliable and comfortable alternative to the fixed structure of the conventional device and other prosthetic leg. The user can with minor training can self-adjust the heel height to the optimum position for comfort and safety based on the particular shoes that he is wearing. Because the present prosthetic ankle is designed to be modular unit, no specialized artificial foot needs to be manufactured. It is more economical to make, and can be integrated into currently existing, commercially available prosthetic leg designs. The attachment of the prosthetic ankle may be accomplished through the use of a commonly available Pyramid fitting system using screws.

The inventive prosthetic ankle comprises of an upper base unit and a lower base unit, which forms the top and bottom interface of the inventive prosthetic ankle assembly for the attachment to the leg and the foot component of a prosthetic leg. In a preferred embodiment, the base units having at least a 2 inches by 2 inches surface area to be suitable for attachment to most commercial prosthetic legs. Each base unit has a plurality of evenly spaced screw holes near the peripheral of the base unit, which can be used to secure the base units onto the top of the wings units using screws and to attach the prosthetic ankle to the leg and foot components of a prosthetic ankle. In an embodiment of preferred operation, long screws are extended through the matching screw hole in both the upper base unit and the upper wing unit to attach the prosthetic leg component to the prosthetic ankle. Similarly, screws maybe inserted into the matching screws holes of both the lower base unit and the lower wing unit to attach the foot component of a prosthetic leg to the prosthetic ankle. The pair of upper or lower wing units is secured evenly apart onto corresponding base unit via holes on top. Each upper wing unit has a pair of raised circular bearing rings are separated by a gap to accommodate the single raised bearing ring of the lower wing unit. The bearing rings are sized and positioned to support an Axle, which connects an upper wing unit to a lower wing unit on the one side and allowing the attached upper base unit and the lower base unit open and close on the other side. Transverse to the base unit is a pair of shear pin holes, which are sized and adapted for an axle that act as the bearing. Optional shearing pins may be inserted into recesses in the base unit to further ensure alignment of the prosthetic leg components. Turnbuckle assembly comprises of two female units and a male unit assembly. The two female units are identical except the reverse interior threads in one end. The male unit has a raised turn wheel in the center, and external threads on both ends, each of which matches the interior thread of one of the female unit. The unthreaded end of the female units contains a semicircular or circular cutout/hole, which is sized to grasp the circumference of the adjustment pins of the base unit. The adjust pin unit rest on the base units, which provide the present invention with its user-adjustability feature. When the user turns a raised wheel in the center of the Male unit, the Male/Female assembly will expand or contract like a turnbuckle, changing the length of the turnbuckle assembly, and thus angle between the upper base and lower base units and compensating for the pitch angle desired. The sliding contact between the Female units and the Adjustment pins prevents any binding or torque as the invention changes angles.

Axle, Shear pin and the Adjustment pins used in the present invention is important load bearing components for the prosthetic ankle. They Adjustment Pins and the Axle all have small grooves on their surfaces to allow circular rings to attach and hold the pins or axle in place in their respective bearings by cotter pins or C-Clips on their ends.

### BRIEF DESCRIPTION OF THE DRAWINGS

FIG. 1 provides rear schematic view of an embodiment of the inventive prosthetic ankle.

FIG. 2 provides a front schematic view of an embodiment of the inventive prosthetic ankle.

FIG. 3 provides a side schematic view of an embodiment of the inventive prosthetic ankle.

FIG. 4 provides a top schematic view of an embodiment of the inventive prosthetic ankle.

FIG. 5 provides close up schematic view of an embodiment of the inventive prosthetic ankle.

FIG. 6 provides a schematic view of the bottom of an embodiment of the base unit.

FIG. 7 provides four views of an embodiment of the base unit.

FIG. 8 provides another four views of an embodiment of the base unit.

FIG. 9 provides a partial schematic view of an embodiment of the turnbuckle assembly.

FIG. 10 provides four views of an embodiment of the female unit of a turnbuckle assembly.

FIG. 11 provides four views of an embodiment of a male unit of a turnbuckle assembly.

FIG. 12 provides five views of an embodiment of the Lower Wing Unit.

FIG. 13 provides cross-sectional schematic view of an embodiment of the Lower Wing Unit.

FIG. 14 provides four views of an embodiment of the Upper Wing Unit.

FIG. 15 provides cross-sectional schematic view of an embodiment of the Upper Wing Unit.

FIG. 16 provides views of the Axle, and the Shear Pins.

### DETAILED DESCRIPTION OF THE INVENTION

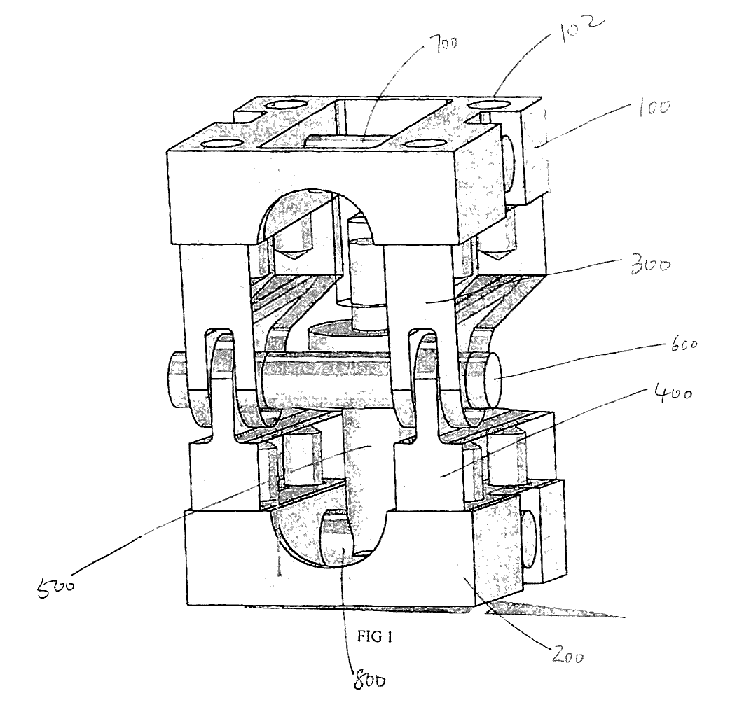

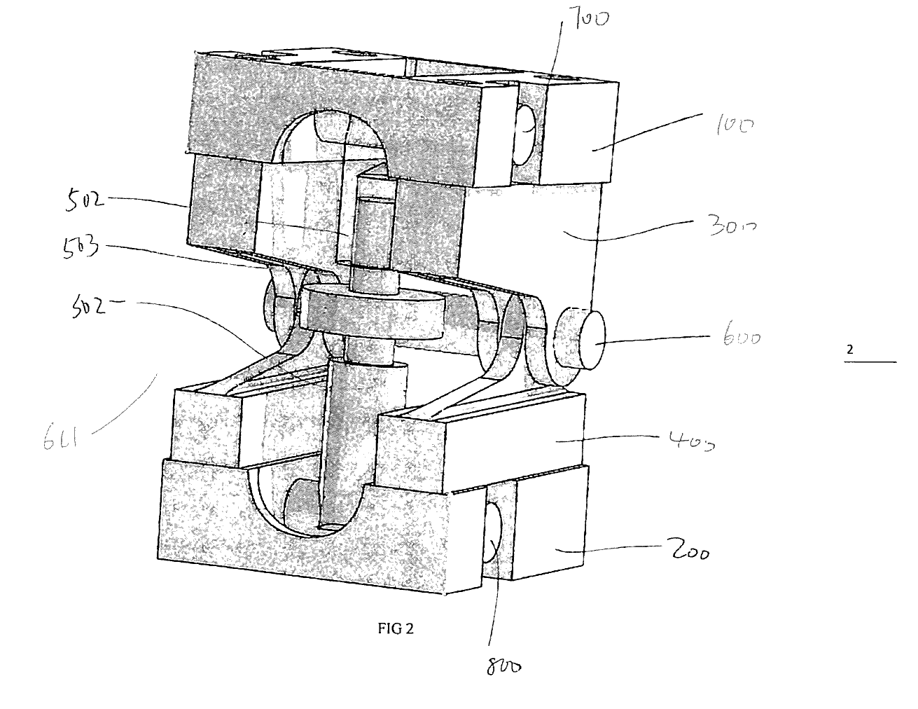

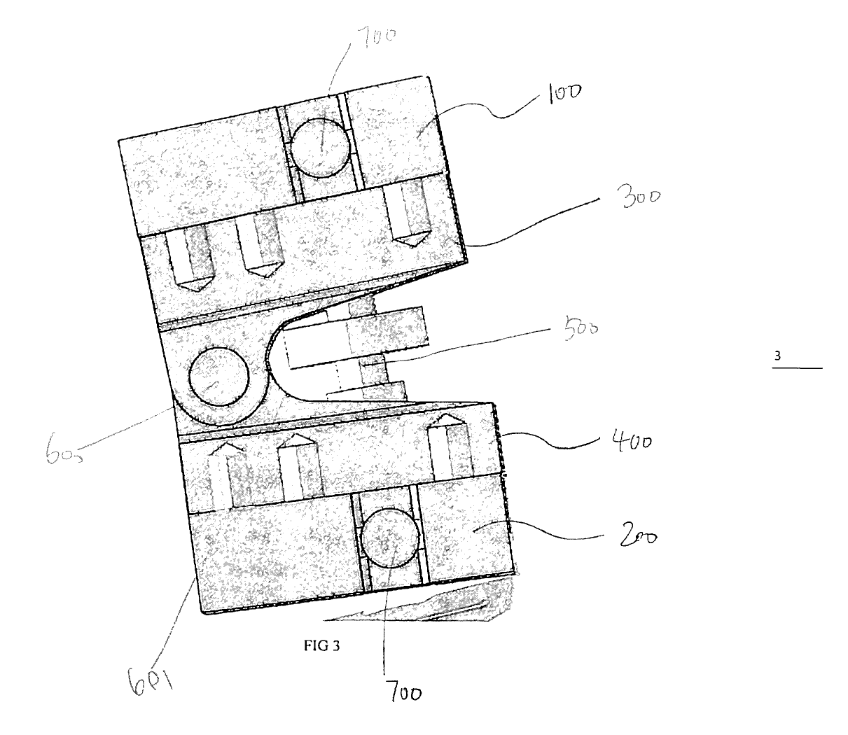

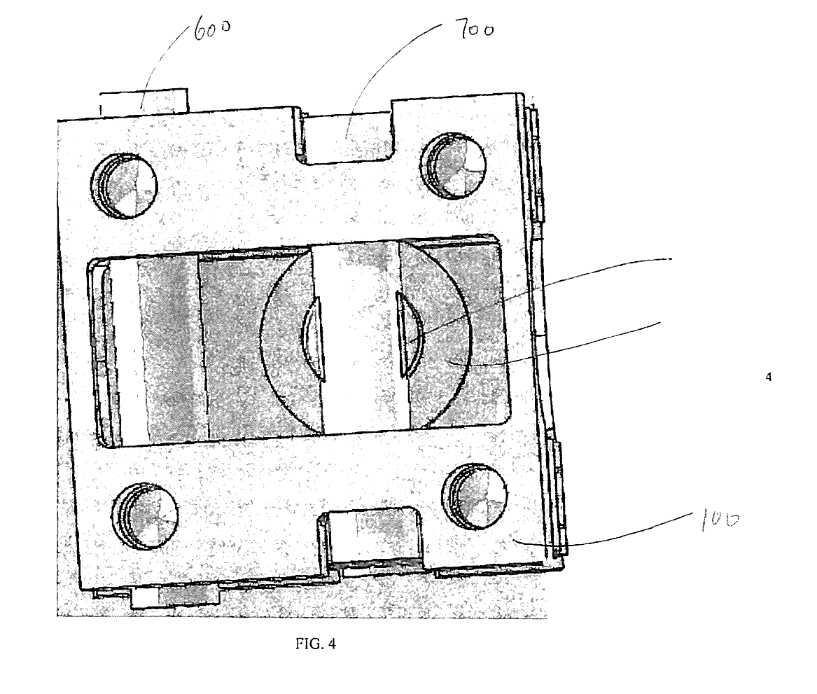

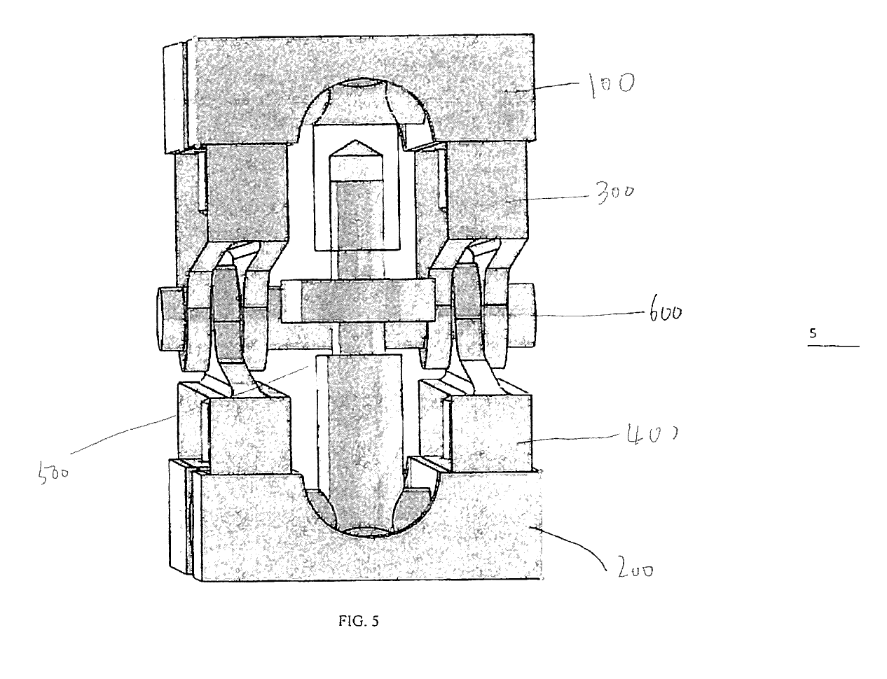

Referring to FIG. 1-5, a prosthetic ankle of the present invention comprises an upper base unit 100 for rigidly connecting to the leg, shin or stump component of a prosthetic leg; a lower base unit 200 for rigidly connecting to the foot component of a prosthetic leg; a pair of upper wing units 300, which are rigidly connected to the upper base unit 100; a pair of lower wing units 400 that are rigidly connected to the lower base unit 200. The upper 300 and lower wing 400 units are engaged and restrained to each other on one side, while allowing rotational movement relative to each other the opposite side. The upper and lower base units have a top surface and bottom surface, whose area is at least two inches by two inches. The upper and lower base unit also have a thickness, which forms a continuous side. A turnbuckle assembly 500 is disposed between the upper base unit 100 and the lower base unit 200, which can extend or contract to change the angle between the upper base unit and the lower base unit and in turn the pitch angle of a prosthetic leg connected to the prosthetic ankle.

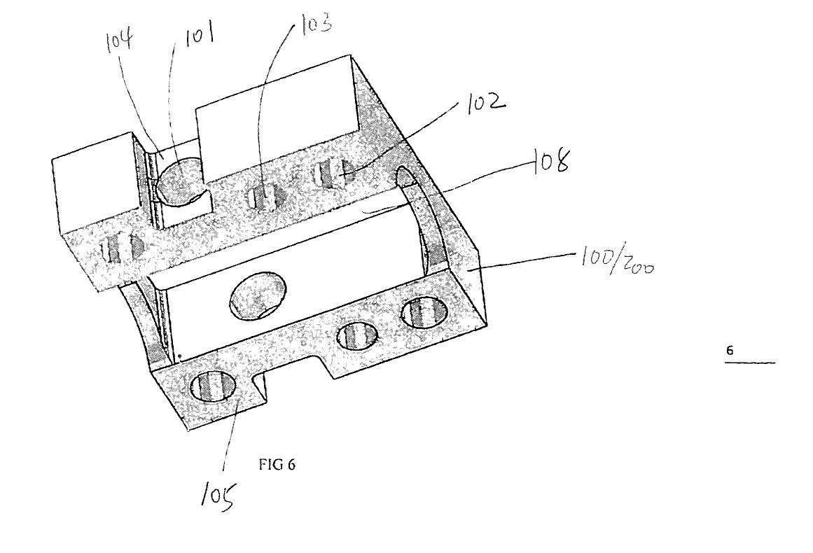

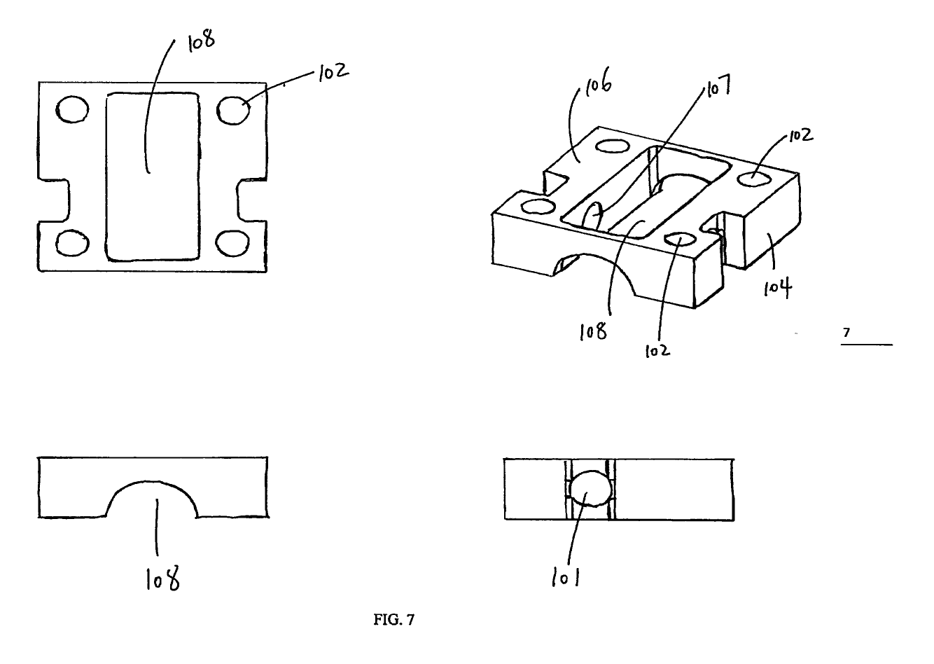

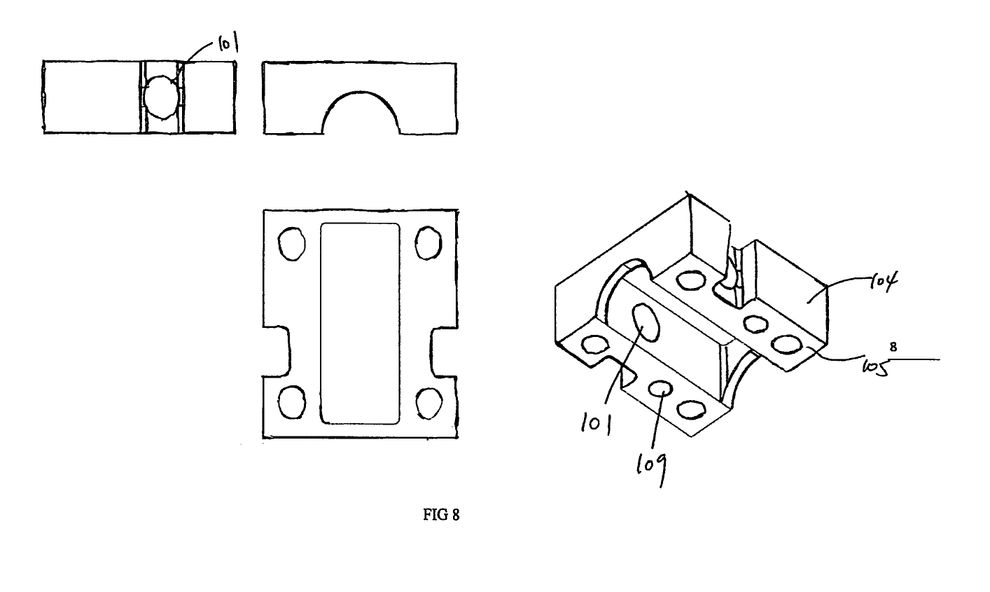

Referring to FIG. 6-8, an embodiment of the upper and lower base units may comprises of a top surface 106, a bottom surface 105 and a side 104. The base units may contain a plurality of evenly spaced holes 102 near the peripheral of each base unit. These threaded holes 102 ensure the rigid connection between the base unit and the corresponding wing unit using screws. Long screws from the Pyramid fittings of a prosthetic leg may be extended through the screw holes in the base unit into corresponding screws holes on the wing units and thus connect them together. The base units may contain cutouts 108 to reduce the overall weight of the prosthetic ankle without comprising its integrity or strength. An additional hole 101 may be drilled through the side of the base unit to allow the insertion of an adjustment pin 700/800, which serves as the bearing for the turnbuckle assembly. The base units may contain one or more additional blind holes 103/109 drilled into the surface facing each other, which acts as matching recesses on the bottom surface of the upper base unit and top surface of the lower base unit, both sized to receive a shearing pin, which once inserted further ensures the alignment of the base units and in turn the vertical alignment of the leg component to the ankle component of a prosthetic leg.

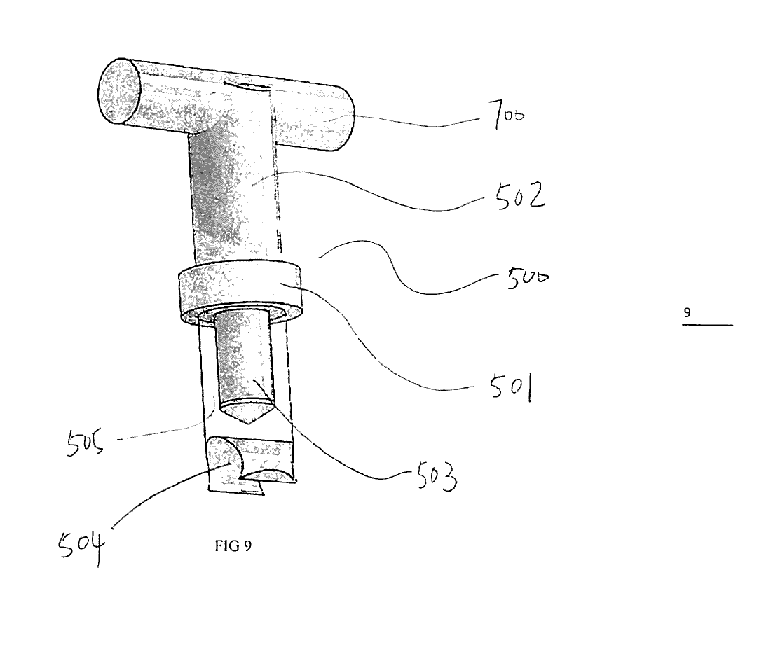

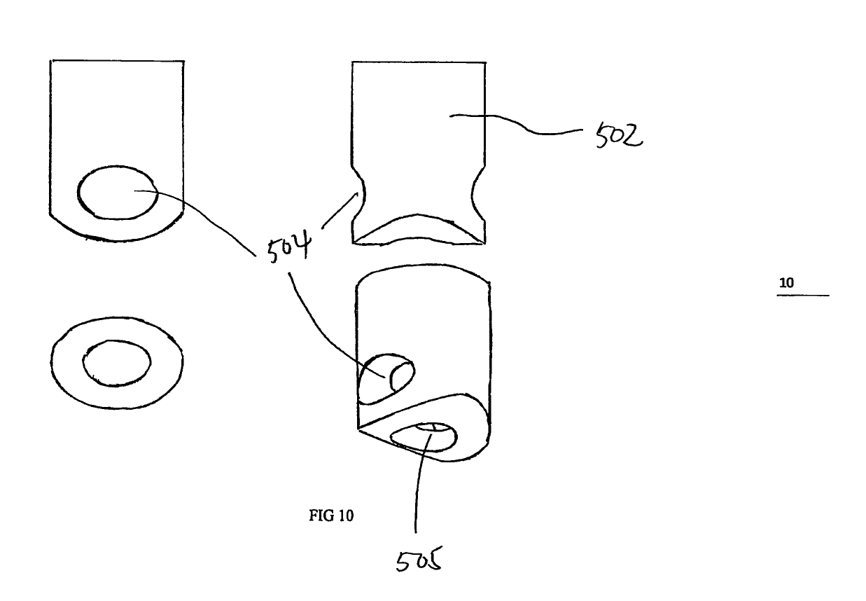

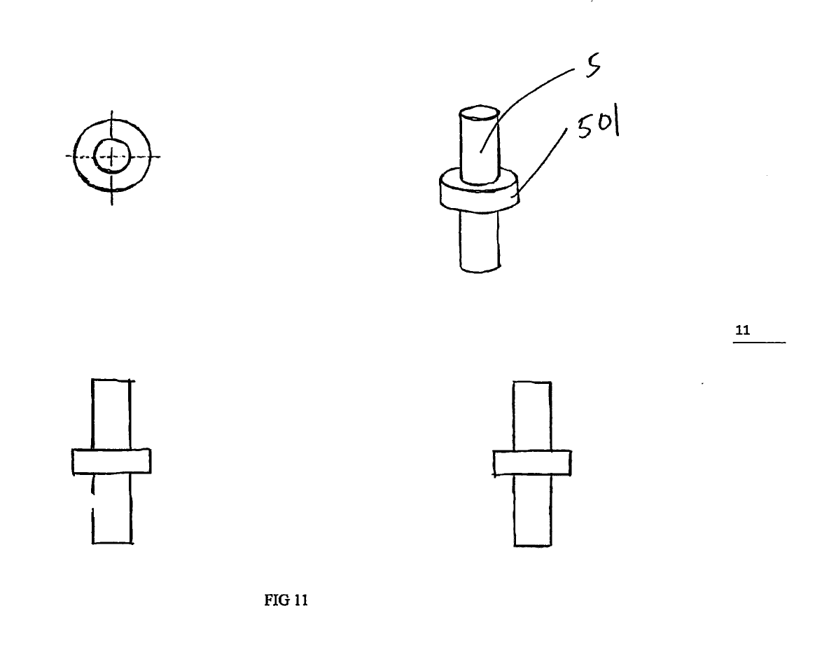

Referring to FIGS. 1-5 and 9-11, a turnbuckle assembly 500 comprises of two female units 505 and a male unit 503. The two female units 505 may be identical but have the interior threads in opposite directions. The male unit 503 has a raised turn wheel 501 rigidly connected to it in the center, and exterior threads on both ends (see FIG. 11), each set of threads matches the interior threads of one of the female units, and locks the female units and the male unit in place (see FIG. 9). One end of the female units may contain a semicircular (see FIG. 9) or circular (FIG. 10) cutout 504, which is sized to grasp the adjustment pins 700/800 of the base unit by its circumference (see FIG. 9). A turnbuckle assembly 500 is locked between the two base units, which provide the present invention with its user-adjustability feature. When the user turns the raised wheel 501 in the center of the Male unit, the Male/Female units will expand or contract like a turnbuckle, changing the length of the turnbuckle assembly, and in turn change the angle between the upper base and lower base units 601. The sliding contact between the

Female units and the adjustment pins 700/800 prevents any binding or torque as the invention changes angles.

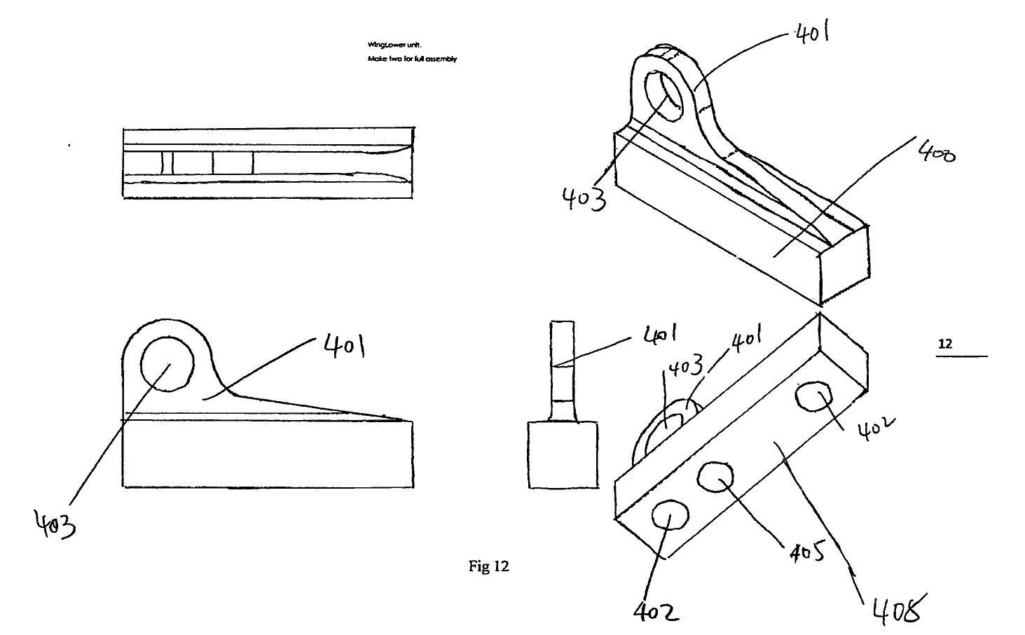

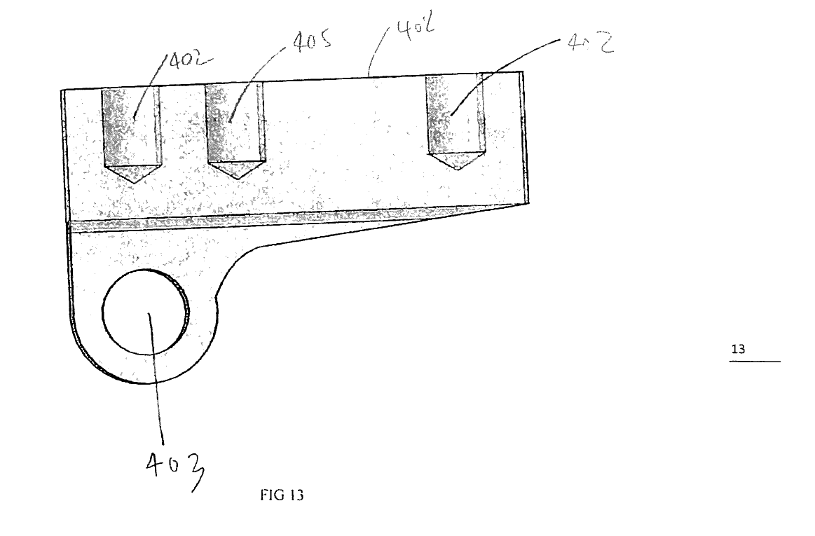

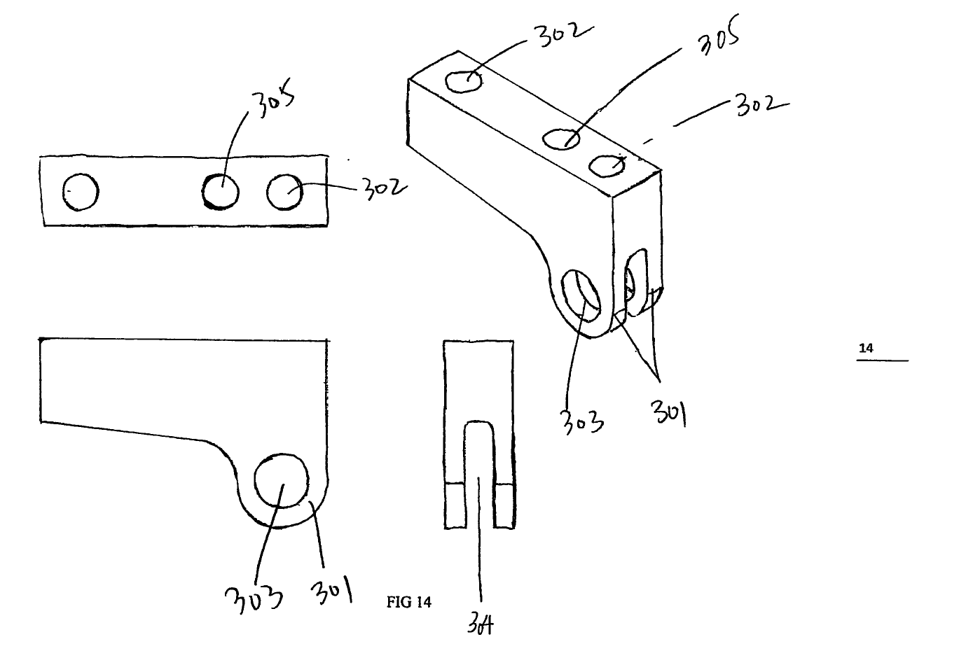

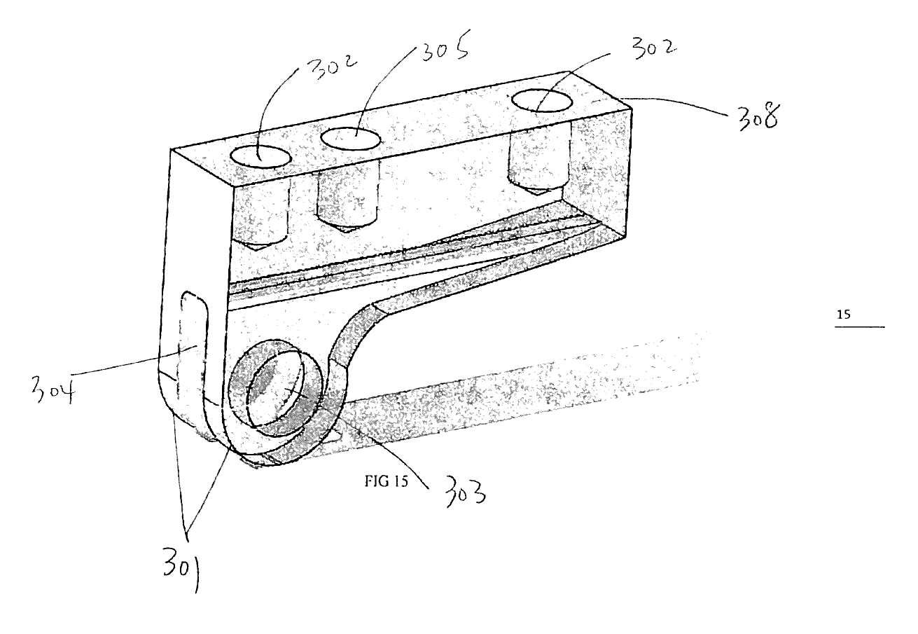

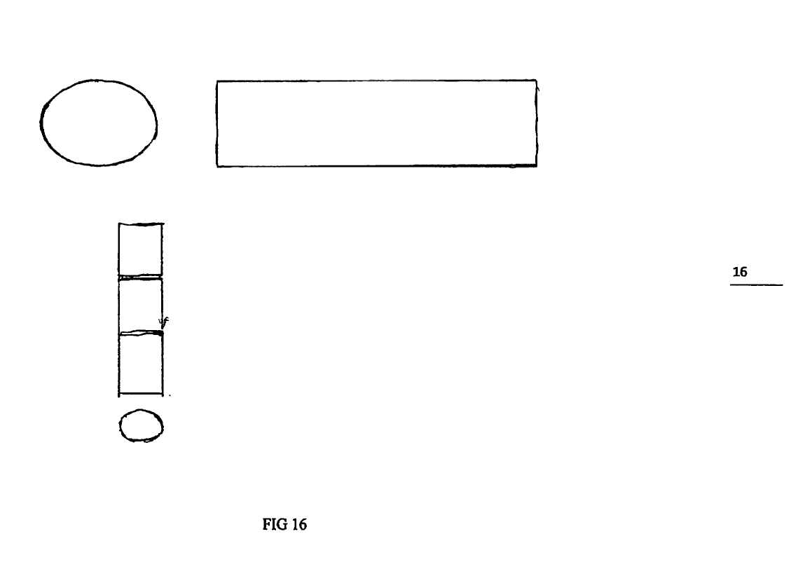

Referring to FIG. 1-5, a pair of upper wing units 300 or a pair of lower wing units 400 is secured evenly apart onto corresponding base unit 100/200. In one embodiment, the wing units are secured to the base unit using screws. Referring to FIG. 14-15, each upper wing unit has a plurality of screw holes 302 evenly spaced on the top 308, which are positioned and sized to receive screws that extended from the matching screw holes 102 of the upper base unit 100. Similarly, referring to FIG. 12-13, each lower wing unit 400 has a plurality of screw holes 402 evenly spaced on the bottom 408, which are positioned and sized to receive screws that extended from the matching screw holes 202 of the lower base unit 200. Each upper wing unit 300 has a pair of raised circular bearing rings 301 are separated by a gap 304 that is sized to receive the single raised bearing ring 401 of the lower wing unit 400. The overlapping bearing rings 303/403 are sized and positioned to support an Axle 600, which engages the upper wing units to the lower wing units on the one side of the base units, while allowing the attached upper base unit 100 and the lower base unit 200 to freely open and close on the other side (see FIG. 1-5) to any angle between −5 degrees and 30 degrees. FIG. 16 shows embodiment of an axle 600 or adjustment pins 700/800, which may have small grooves/circular notches on their surfaces to allow circular rings to attach, and hold the axles/adjustment pins in place within its bearing. The adjustment pins 700/800 and axles 600 are retained in their respective bearing by applying cotter pins or C-Clips on their ends.

### Operation

The operation of the described embodiment of the prosthetic ankle of the present invention is believed to be clearly apparent and is briefly summarized at this point. Initially, the foot components of a prosthetic leg using commercially available Pyramid fittings that is compatible to many prosthetic leg systems. The foot is fitted into the shoe that user plan to wear. The pitch angle is then adjusted for by turning the raised wheel with a thumb to extend or contract the turnbuckle. In a preferred embodiment, the pitch angle may be adjusted to any angle between −5 degrees (toe up) to 30 degrees (ankle up) by turning a wheel with a thumb. No additional tool is required for the adjustment. The pitch angle is locked in place by match threads of the female and male units of the turnbuckle assembly. This design is located higher on the leg, reducing the swinging momentum and enabling a smoother gait by the user.

### Testing

Human testing focus on the utility of the design. This includes the ease of test subjects to manually operate the adjustment of the device, and the effects on the gait and posture. Various combinations of shoe heel heights and subjects of different gender, body weight, age and different leg prosthetic will be evaluated in the specialized Gait Analysis Laboratory at the Naval Medical Center San Diego (NMC SD).

Simulated stress and fatigue tests will be conducted by standardized mechanical design software. Technical analysis will include inspection of the unit for wear and strength performance after a period of use by a test participant. Laboratory testing of the materials and architecture of the design will be conducted using a universal testing machine also at NMCSD. Loading and fatigue performance will be evaluated in accordance with the International Standard for Ankle-Foot devices (ISO 22675).

What is claimed is:

1) A prosthetic ankle for connecting an artificial leg with an artificial foot, said prosthetic ankle and adjusting its pitch angle comprising:
a) an upper base unit for rigidly connecting to said artificial leg, having a top surface, a bottom surface and a side;
b) a lower base unit for rigidly connecting to said artificial foot, having a top surface, a bottom surface and a side;
c) a pair of lower wing units having a bottom surface for rigidly connecting to said top surface of said lower base unit, wherein said lower wing units are disposed evenly apart;
d) a pair of upper wing units having a top surface for rigidly connecting to said bottom surface of said upper base unit, wherein said upper wing units are engaged and restrained to said lower wing units on one side to allow rotational movement of said upper and lower base units on the other side; and
e) a turnbuckle assembly loosely connected between the bottom surface of the upper base unit and the top surface of the upper base unit.

2) The prosthetic ankle of claim 1, wherein said upper and lower base units have a top and bottom surface area at least 2 inch by 2 inch.

3) The prosthetic ankle of claim 2, wherein said upper and lower base units have a plurality of screw holes disposed near the peripherals of said upper and lower base units

4) The prosthetic ankle of claim 3, wherein said upper and lower wings units are rigidly connected to respective upper and lower base units by extending screws through said screw holes and into the wing units.

5) The prosthetic ankle of claim 3, wherein said prosthetic ankle is connected to the artificial leg and artificial foot by extending screws through said screws holes.

6) The prosthetic ankle of claim 1, wherein said upper base unit and upper base wing units, and said lower base unit and lower base wing units contains at least one blind hole for insertion of bearing pins.

7) The prosthetic ankle of claim 1, wherein said upper and lower base units having one or more cutouts.

8) The prosthetic ankle of claim 7, wherein an adjustment pin is locked inside the cutout of each base unit for serving as bearing for said turnbuckle assembly.

9) The prosthetic ankle of claim 8, where a turnbuckle is connected to said adjustment pin to allow rotational movement of the turnbuckle around the adjustment pin.

10) The prosthetic ankle of claim 1, wherein said turnbuckle comprising of
a) a pair of identical cylindrical female unit with opposing interior threads;
b) a cylindrical male unit having opposing exterior threads on each end, whereas the male unit is inserted into and locked inside each of said female units by matching the exterior threads to the interior threads of the female units.

11) The prosthetic ankle of claims 8 and 9, wherein a semicircular or circular hole is drilled on one end of said female unit perpendicular to the cylindrical body, and is sized and adapted to receive an adjustment pin.

12) The prosthetic ankle of claim 10, wherein the male unit having a turning wheel in the center.

13) The prosthetic ankle of claim 4, wherein said upper wing unit has a pair of raised bearing ring spaced to receive a single raised bearing ring of said lower wing unit.

14) The prosthetic ankle of claim 12, wherein each pair of said upper wing unit and lower wing unit are connected by an axle inserted through said raised bearing rings.

15) The prosthetic ankle of claim 1, wherein said pitch angle may be set between −5 degrees and 30 degrees.

16) A method for adjusting for the pitch angle of a prosthetic leg, comprising
a) attaching a prosthetic ankle of claim 1 to a prosthetic leg;
b) placing an artificial foot of said prosthetic leg into a shoe; and
c) adjusting the length of said turnbuckle assembly to adjust the pitch angle of a prosthetic leg.

17) The method of claim 16, where pitch angle may be adjusted between −5 degrees and 30 degrees.
